(19) United States
(12) Patent Application Publication
(10) Pub. No.: US 2003/0170328 A1
(43) Pub. Date: Sep. 11, 2003

(54) Anti-inflammatory formulations

(76) Inventors: David Haines, McClean, VA (US); Fadia F. Mahmoud, Salawa (KW)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/345,856

(22) Filed: Jan. 16, 2003

Related U.S. Application Data
(60) Provisional application No. 60/350,298, filed on Jan. 16, 2002.

(51) Int. Cl. A61K 35/78; A61K 31/366; A61K 31/375; A61K 31/203; A61K 31/07

(52) U.S. Cl. 424/752; 514/559; 514/474; 514/460; 514/725; 514/552

(57) ABSTRACT

The invention features compositions containing an antioxidant and/or a gingkolide compound to reduce inflammation. Combination drug therapy using antioxidant and/or a gingkolide compound with an anti-inflammatory agent reduces adverse side effects associated with many known anti-inflammatory agents.

This application claimed priority to provisional application U.S. Ser. No. 60/350,298, filed on Jan. 16, 2002, the entire contents of which is hereby incorporated by reference.

### BACKGROUND OF THE INVENTION

The invention relates to control of inflammation.

Anti-inflammatory drugs such as corticosteroids have been used clinically to treat both chronic and acute inflammation associated with a diverse range of disease. In many cases, adverse side effects such as decreased cell-mediated immunity result from their use, particularly if it is long term as in asthma or psoriasis. This limits their clinical benefits and reduces their usefulness.

### SUMMARY OF THE INVENTION

The invention features an anti-inflammatory composition, which is associated with reduced adverse side effects such as decreased cell-mediated immunity compared to conventional anti-inflammatory drugs. The anti-inflammatory composition contains a lipid-soluble antioxidant carotenoid. In some embodiments, the composition does not contain a beta-carotene compound. The composition may also contain a water-soluble antioxidant (vitamin C or ascorbic acid) and/or a ginkgolide.

Accordingly, the invention provides a composition containing a lipid soluble antioxidant and a water soluble antioxidant. The lipid soluble antioxidant is a carotenoid compound. The carotenoid compound is astaxanthin or an ester thereof or a vitamin such as ascorbic acid. Alternatively, the water soluble antioxidant is a ginkgolide such as a terpene trilactone selected from the group consisting of Gingkolide A, Gingkolide B, Gingkolide C, Gingkolide J, Gingkolide M, and bilobalide. The gingkolide composition preferably exhibits one or more of the following activities: (i) platelet activating factor receptor (PAFR) antagonist activity; (ii) PLA2-inhibitory capability; (iii) COX-2-inhibitory capability; and (iv) the capability to inhibit cAMP phosphodiesterase. Preferably, the composition exhibits all of the aforementioned activities. For example, the ginkgolide composition contains Egb 761. The composition optionally also contains an histamine release inhibitor such as a cetirizine compound and/or an azelastine compound. In preferred embodiments, the composition contains a mixture of an asaxanthin compound, a ginkgolide compound, and an ascorbic acid compound.

The invention also includes a method of suppressing inflammation in a mammal. The method is carried out by co-administering of astaxanthin or a derivative thereof, vitamin C, and one or more classes of gingkolide in such amounts so as to provide an additive or synergistic anti-inflammatory effect. Preferably, the gingkolide is administered at a dose that preferentially inhibits expression of an inflammatory cytokine much as IL-8, IL-1α, IL-1β, TNF-α or IL-6.

For example, the invention provides a method of inhibiting activation of an immune cell by contacting the immune cell (e.g., a T cell or a mast cell) with the composition(s) described above. Also within the invention is a method of alleviating a symptom of an inflammatory disease by administering to a mammal suffering from or at risk of developing the disease one or more of the anti-inflammatory composition described above. In one example, the composition is administered systemically. Alternatively, the composition is administered locally. For example, the composition is administered by directly contacting an inflammed tissue with the composition. The tissue to be directly contacted is dermal tissue in the case of skin inflammatory diseases such as psoriasis. For asthma, the tissue is pulmonary tissue, e.g., bronchoalveolar tissue. In the former case, the compositions are administered topically, e.g., by contacting skin with a cream, lotion, or ointment. In the latter case, pulmonary tissue is contacted by inhaling a composition, e.g., a liquid or powder aspirate containing the mixture of anti-inflammatory compounds.

Antioxidants such as carotenoids are co-administered with other agents to reduce inflammation. For example, astaxanthin (or esters thereof), vitamin C, and the gingkolide(s) are administered simultaneously or consecutively. For example, the gingkolide(s) is first administered followed by astaxanthin, followed by vitamin C. Alternatively, astaxanthin is administered first and then the gingkolide(s) and then vitamin C. In another regimen, vitamin C is administered first, followed by astaxanthin, followed by the ginkgolide(s); or vitamin C is administered following administration of either astaxanthin or the ginkgolide, followed by administration of the third component. The combination of compounds is administered in the presence or absence of a traditional anti-inflammatory agent such as a corticosteroid or non-steroidal anti-inflammatory agent.

Such a co-administration regimen is useful to inhibit inflammation in a mammal. For example, each of the aforementioned thre classes of compounds are administered prior to after development of inflammation as a prophylaxis; or after development of inflammation as a therapeutic.

The antioxidant and gingkolide compounds described are also useful in combination with nonsteroidal anti-inflammatory drugs (NSAIDs) to reduce the dose of NSAID required to achieve a desired clinical effect such as reduction of symptoms associated with Alzheimer's Disease. Combined with histamine release blockers such as cetirizine, the antioxidant and gingkolide compounds augment the clinical effect (e.g., reduction of allergy symptoms such as itching) of the histamine release blocker, thereby permitting administration of a lower dose of the histamine release blocker. Coadminstraion of an antioxidant and/or a gingkolide compound reduces adverse side effects associated with many known anti-inflammatory and anti-allergy medications.

### BRIEF DESCRIPTION OF THE DRAWINGS

FIG. 1 is a bar graph showing the effect of astaxanthin (ASX) on immune activation of human PBMC. Cells cultured 24 h at 37° C., 5% CO₂ in RPMI 1640, 10% FCS with 50 mg/ml PHA and ASX were evaluated by 3-color flow cytometry for immune activation as %CD3+ cells induced to express membrane-bound CD25 (IL-2 receptor). Stimulation indices (SI) were determined as the ratio of %CD3+CD25+ cells in fully-stimulated cultures treated with PHA alone, to those cultured with PHA plus ASX. Results are representative of independent assays conducted on cells of 6-8 asthmatic donors participating in this study. Significance in comparison with fully-stimulated cultures: (*: p<0.05)

FIG. 2 is a bar graph showing the effect of ginkgolide B (GB) on immune activation of human PBMC. Cells cultured 24 h at 37° C., 5% CO₂ in RPMI 1640, 10% FCS with 50 mg/ml PHA and GB were evaluated by 3-color flow cytometry for immune activation as %CD3+ cells induced to express membrane-bound CD25 (IL-2 receptor). Stimulation indices (SI) were determined as the ratio of %CD3+CD25+ cells in fully-stimulated cultures treated with PHA alone, to those cultured with PHA plus GB. Results are representative of independent assays conducted on cells of 6-7 asthmatic donors participating in this study. Significance in comparison with fully-stimulated cultures: (*: p<0.05)

FIG. 3 is a bar graph showing the effect of astaxanthin (ASX) plus ginkgolide B (GB) on immune activation of human PBMC. Cells cultured 24 h with 50 mg/ml PHA and selected combinations of ASX+GB were evaluated by 3-color flow cytometry for immune activation as %CD3+ cells induced to express CD25 (IL-2 receptor). Stimulation indices (SI) are determined as the ratio of %CD3+CD25+ cells in fully-stimulated cultures treated with PHA alone, to those cultured with PHA plus selected combinations of ASX+GB. Results are representative of independent assays conducted on cells of 4-7 healthy adult donors participating in this study. Significance in comparison with fully-stimulated cultures: (*: p<0.05)

FIGS. 4A-4B are bar graphs showing the effect of cetirizine (Zyrtec/CTZ) versus azalestene (AZE) on immune activation of human PBMC. Cells cultured 24 h at 37° C., 5% CO₂ in RPMI 1640, 10% FCS with 50 mg/ml PHA and either CTZ (4A) or AZE (FIG. 4B) are evaluate by 3-color flow cytometry for immune activation as % CD3+ cells induced to express membrane-bound CD25 (IL-2 receptor). Results are representative of independent assays conducted on cells of 7-12 asthmatic donors. Significance (P) in comparison with fully-stimulated culture: (*:p<0.05)

### DETAILED DESCRIPTION

The compositions described herein are useful to prevent inflammation, and improve the clinical prognosis for patients suffering from inflammatory disease. The combined action of a lipid-soluble carotenoid (principally astaxanthin) with vitamin C and one or more components of a Ginkgo biloba extract mediates prevention or suppression of disease-associated inflammation.

Astaxanthin

Astaxanthin (3,3′-dihydroxy-4,4′-diketo-β-carotene) is a carotenoid produced by several natural sources, including: the marine algae Haematococcus pluvialis; and the pink yeast Xanthophyllomyces dendrorhous. It is obtained directly from either aforementioned organism; or alternatively by extraction from by-products of crustacea such as the Antarctic krill Euphausia superba. Its molecular structure is similar to that of carotenoid beta-carotene, however small differences in structure confer large differences in the chemical and biological properties of the two molecules. In particular, astaxanthin is superior to beta-carotene in its capacity to scavenge free radicals. It exhibits strong antioxidant properties and confers protection against lipid peroxidation and oxidative damage of LDL-cholesterol, cell membranes, cells, and tissues. Beneficial effects mediated by astaxanthin in mammals are known to include: increased boar semen volume and piglet litter size and survival rate when fed to pigs; augmentation of anti-stress agents administered to farm animals and household pets; improved immunity; and suppression of tumor growth.

Additionally, esterified astaxanthin from Haematococcus pluvialis algal meal is therapeutic for muscular dysfunction such as exertional rhabdomyolysis (also known as exertional myopathy, tying-up syndrome, azoturia, or Monday morning sickness) in horses; and for gastrointestinal tract inflammation due to infections by Helicobacter sp. bacteria.

Gingkolides

Ginkgo Biloba. is a plant, the leaves, roots, and fruit of which have been used for medicinal purposes for centuries. Extracts of various parts of the plant are commercially available. A gingkolide, or Ginkgo biloba extract contains one or more biologically active components such as an antioxidant component and an PAFR antagonist component. For example, an extract is made from ginkgo leaves and used at a concentration that contains about 24-25% ginkgo-flavone-glycosides. The extract may also contain terpenoids such as Egb761 or LI-1370. For example, the preparation contains 24% ginkgo-flavone glycosides and 6% terpenoids. The ginkgo-flavone glycosides are sometimes referred to as heterosides. EGb761 is a commercially available leaf extract of Ginkgo biloba, containing: GA, GB, GC, GJ, GM and bilobalide.

Naturally-occurring Ginkgo biloba contains: (A) biflavones such as amentoflavone, bilobetin, sequoiaflavone, ginkgetin, isoginkgetin, Sciadopitysin; (B) flavonol glycosides; (C) terpene trilactones, such as Gingkolide A, Gingkolide B, Gingkolide C, Gingkolide J, Gingkolide M and bilobalide; (D) rutin; (E) quercetin; and (F) a 30 kDa Ginkgo biloba glycoprotein, which reacts with antiserum against beta 1→2 xylose-containing N-glycans. Each component or combinations thereof are isolated from crude extracts of the plant using methods known in the art.

Alphabetically-labeled series of ginkgolide derivatives are further characterized as follows. Ginkgolide A (GA) is a leaf extract containsin terpene trilactone. This gingkolide is a PAFR antagonist, but has no apparent antioxidant properties. It is also known as BN52020, CAS 15291-75-5. Ginkgolide B (GB) is a leaf extract containing terpene trilactone. It is a PAFR antagonist, with antioxidant properties and may be referred to as BN52021 or CAS 15291-77-7. GC, ginkgolide C: a terpene trilactone, leaf extract. A PAFR antagonist, with antioxidant properties. Ginkgolide J (GJ) is a leaf extract containing terpene trilactone with PAFR antagonist activity and antioxidant properties. Ginkgolide M (GM) is a root extract containing terpene trilactone. This gingkolide has PAFR antagonist activity and antioxidant properties. Bilobalide (a sesquiterpene trilactone) is primarily an antioxidant. Ginkgo biloba extract (EGb 761) is a clinically safe, nontoxic, and easily-produced product with a wide range of applications.

Other extracts and preparation of gingkolides are known in the art, e.g., as described in Chen et al., 1998, Bioorganic & Medicinal Chemistry Letters 8:1291-6.

The gingkolide compositions to be administered are in a form which maximizes ginkgolide bioavailability. For example, the composition is a variation of EGb 761 containing 27% ginkgo-flavonol glycosides, 7% terpene lactones. This composition extends bioavailability of pharmacologically active ginkgolide components (Li et al, 1997, Planta Medica. 63:563-5).

Among the compositions to be administered is BN 50730, an analog to the terpene trilactone BN52021 (GB). BN 50730 is a synthetic hetrazepine derivative of BN 52021. It shows a several ten-folds more potent PAF antagonistic activity in vitro than BN52021.

Anti-inflammatory Drug Combinations

The dose-response curve of astaxanthin in suppression of in vitro expression of an inflammation-associated cytokine was found to be favorably altered in the presence of a ginkgolide. Inflammatory damage is suppressed by astaxanthin or its derivatives and further reduced by co-administration of a ginkgolide.

The combination drug therapy regimen described herein is based on the pharmacological action of astaxanthin, ginkgolides and vitamin C. By acting as a powerful scavenger of free radicals, astaxanthin inhibits tissue damage mediated by these chemical species. However, since astaxanthin and its derivatives are primarily lipid-soluble, the adduct often remains membrane associated. Effective clearance of free radical-astaxanthin reaction products is mediated by co-administration of a water-soluble scavenger of free radicals. For example, the water-soluble free radical scavenger is vitamin C. Gingkolide compositions include extracts of ginkgo such as EGb761. The gingkolide alone or in combination with vitamin C; or astaxanthin alone, or in combination with vitamin C; or astaxanthin plus a ginkgolide; or astaxanthin plus a ginkgolide plus vitamin C are used for suppression of disease-associated inflammation.

For example, the dose of astaxanthin plus ginkgolide and vitamin C required to achieve clinically significant suppression of inflammation is at least 5%, preferably at least 10%, preferably at least 25%, preferably at least 30%, more preferably at least 40%, and most preferably at least 50% less than that required for the same level of suppression of inflammation in the absence of a gingkolide and vitamin C. Suppression of inflammation is measured using methods known in the art, e.g., by detecting reduced expression of pro-inflammatory cytokines both in vitro (cell culture approach) and in vivo (immunohistochemical approach), given stimulus of experimental model in a manner known in the art to induce expression of these cytokines.

Toxicity

An astaxanthin/ginkgolide/vitamin C combination drug offers a method for achieving suppression of disease-associated inflammation in a manner superior to currently available drugs. Moreover, since each component exhibits low-to-negligible toxicity levels, and therefore, are applicable to a broad patient population.

Treatment and Alleviation of Symptoms of Inflammatory Disease

Clinical effects of formulations based on co-administration of astaxanthin plus ginkgolides and/or vitamin C include application to inflammation associated with autoimmune conditions (such as type I diabetes), asthma, psoriasis and cardiac disorders. These combinations will also aid in post-organ transplant drug therapy. Suppression of graft rejection-associated inflammation by these drugs is sufficient to maintain transplanted tissue in a healthy, functional state with little or no side effects.

Advantages of the invention include improved outcomes to transplant surgery (both in terms of survival as well as drug-related morbidity), decreased need for secondary hospitalization, and reduced expenditure of health care costs for transplant recipents.

The coadministration strategy also decreases the incidence of ischemia/reperfusion-related damage to organs occurring postoperatively, or as a result of ischemic disease as a result of the capacity of these formulations to inhibit basic inflammatory processes.

Platelet Activating Factor (PAF)/Calcium-dependent Protection and Mechanisms of Inflammation

Cellular signaling pathways resulting in inflammatory responses are dependent largely upon receptor-mediated release of calcium stores (such as within the endoplasmic or sarcoplasmic reticulum), followed by expression of inflammatory mediators. This calcium availability may be reduced by treatment of a subject one or more subcomponents of Ginkgo biloba (e.g., EGb761). The gingkolide acts as an antagonist to the receptor for PAF, a potent bioactive phospholipid. The PAFR, when engaged by PAF, activates a signalling pathway causing a rise in intracellular calcium. Gingkolide compounds inhibit PAF-mediated increase in cytoplasmic calcium, in turn suppressing release of eicosonoids, pro-inflammatory cytokines, free radical species and other major mediators of inflammation.

Prevention of PAF/COX-2-Mediated Effects

PAF stimulates transcription of COX-2 (inducible prostaglandin synthase) which contributes to inflammatory damage. Ischemia of any tissue promotes PAF overproduction. PAF activity is blocked with ginkgolides exhibiting PAF receptor antagonist properties.

Amplification of Pharmacological Effect by Increasing Ginkgolide Bioavailability

EGb 761 is a standardized extract of dried leaves of Ginkgo biloba containing 24% ginkgo-flavonol glycosides, 6% terpene lactones (24/6) such as ginkgolides A, B, C, J and bilobalide. The PAFR antagonistic and antioxidant effects of EGb761 confers clinical benefit, alone or when combined with astaxanthin and/or vitamin C. For example, an immunosuppressive compound contains a calcineurin inhibitor with extract of Ginkgo biloba with a ratio of 27% ginkgo-flavonol glycosides, 7% terpene lactones (27/7), enriched in ginkgolide B. Preparation of the gingkolide portion of the composition is known in the art, e.g., the method of Li, et al., 1997, Planta Medica. 63(6):563-5.

Therapeutic Administration

The results suggest that the combination of astaxanthin and a gingkolide is useful to inhibit inflammatory damage occurring as a result of a diverse range of diseases. The compositions are formulated into therapeutic compositions such as liquid solutions or suspensions, tablets, pills, powders, suppositories, polymeric microcapsules or microvesicles, liposomes, and injectable or infusible solutions. The preferred form depends upon the mode of administration and the particular indication targeted. The compositions also include pharmaceutically acceptable vehicles or carriers. Suitable vehicles are, for example, water, saline, dextrose, glycerol, ethanol, or the like, and combinations thereof. Actual methods of preparing such compositions are known to those skilled in the art (e.g., Remington's Pharmaceutical Sciences, Mack Publishing Company, Easton, Pa., 18th edition, 1990).

The compositions are administered using conventional modes of delivery including intravenous, intraperitoneal, oral or subcutaneous administration. In addition to systemic administration, the compositions are locally administered, e.g., to the site of inflammation.

The dosages of astaxanthin and of gingkolide and vitamin C may vary depending on the severity and course of the disease, the patient's health and response to treatment, and the judgment of the treating physician.

Astaxanthin, vitamin C and the gingkolide are administered simultaneously or sequentially. Astaxanthin dosages range from 0.1-4.0 g/kg body weight per day; gingkolide compositions are administered in doses of 0.1 mg/kg/day to 1000 mg/kg/day. (e.g., 10 mg/kg/day-60 mg/kg/day); and dosage of vitamin C will include regimens of 1.0-400.0 mg/kg/day. Routes of administration are comparable to those used for immunophilin-binding compounds such as calcineurin inhibitors.

The compositions are administered as prophylaxis to prevent onset of an inflammatory condition, or before or after development of disease. Subjects to be treated include those who have been diagnosed as having a condition characterized by aberrant immune activation (e.g., pathological T cell activation or pathological inflammation), those who are at risk of developing such a condition, and those who have a personal or family history of such a condition. Such aberrant inflammatory events include an asthma attack. Methods for diagnosis are known in the art. For example, the anti-inflammatory compositions are useful to treat or prevent autoimmune disease and/or inflammatory conditions such as asthma, arthritis (e.g., rheumatoid arthritis, arthritis chronica progrediente and arthritis deformans) and rheumatic diseases. Specific auto-immune diseases for which the compositions of the invention may be employed include, autoimmune hematological disorders (including e.g. hemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, psoriasis, Steven-Johnson syndrome, idiopathic sprue, autoimmune inflammatory bowel disease (including e.g. ulcerative colitis and Crohn's disease) endocrine ophthalmopathy, Graves disease, sarcoidosis, multiple sclerosis, primary billiary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.

Individuals to be treated include any member of the class Mammalia, including, humans and non-human primates, such as chimpanzees and other apes and monkey species; farm animals such as cattle, sheep, pigs, goats and horses; domestic mammals such as dogs and cats; and laboratory animals including rodents such as mice, rats and guinea pigs. Preferably, the mammal is not a rodent such as a rat. The compositions and methods are suitatble for treatment of adult, newborn and fetal mammals. Treatment encompasses the prevention of and adverse clinical conditions and the reduction or elimination of symptoms of a disease or adverse clinical condition. An anti-inflammatory composition refers to any composition that suppresses or prevents an undesired inflammatory response, e.g., prevents pain, tissue damage and disfigurement.

The combination drug therapy described herein utilizes astaxanthin and/or its derivatives; and a gingkolide composition, which contains PAFR antagonist activity and antioxidant activity. Preferably, the gingkolide compositions contains at least two antioxidant components of Gingko biloba, e.g., GB, GC, GJ, or GM, rather than one component such as GM alone. For example, the gingkolide composition is Egb761 contains several antioxidant components of Gingko biloba in addition to a component with PAFR antagonist activity. EGb761 contains a full range of antioxidants and PAFR antagonists produced by leaves of the plant.

### EXAMPLE 1

### Administraton Of Astaxanthin Leads To Suppression Of Inflammation

In vitro studies indicate that astaxanthin suppresses expression of inflammation-associated T cell surface antigens in PMA/I-treated human PBMC. Cells isolated from whole blood of healthy volunteers were cultured in 96-well plates (2×10⁶ /ml) for 24 hours in RPMI 1640, with phorbol 12-myristate 13-acetate (PMA: 25 ng/ml) in conjunction with ionomycin, or media; or with PMA/I plus astaxanthin (10⁻⁵ M).

Following incubation, cultured cells were immunofluorescently labeled with monoclonal antibodies specific for CD3 (T lymphocytes) and the cell surface antigens CD25 and CD54 which are known to be upregulated in vivo during both immune activation and during inflammatory processes. Analysis of blood for representation by selected lymphocyte subpopulations was conducted by two-color flow cytometry. Astaxanthin alone significantly inhibited each of the activated T cell phenotypes (Table 1A and 1B).

TABLE 1A
(Subject A)

| Stimulation | % CD3 + CD54 + | % CD3 + CD54 + |
|---|---|---|
| Conditions | cells | cells |
| Unstimulated | 1.7 | 2.7 |
| PMA/I | 56.3 | 66.3 |
| Astx 10⁻⁷ M | 8.4 | 10.6 |
| Astx 10⁻⁶ M | 4.2 | 3.3 |

TABLE 1B
(Subject B)

| Stimulation | % CD3 + CD54 + | % CD3 + CD54 + |
|---|---|---|
| Conditions | cells | cells |
| Unstimulated | 2.2 | 1.6 |
| PMA/I | 49.5 | 54.6 |
| Astx 10⁻⁷ M | 16.2 | 21.9 |
| Astx 10⁻⁶ M | 6.3 | 10.3 |

### EXAMPLE 2

### Expression Of TNF-α By Human PBMC In Vitro Is Suppressed By Astaxanthin But Not BN52021 And Is Suppressed Maximally With Astaxanthin Plus BN52021

PBMC (2×10⁶/ml) 2 donors were stimulated with 50 μg/ml, PHA; or with astaxanthin (10⁻⁶ M); or with the ginkgolide BN52021 (GB) (10⁻⁴ M); or with a combination of astaxanthin (10⁻⁶ M) plus GB (10⁻⁴ M); or with media. Cells were cultured 24 hours at 37° C., CO₂ and analyzed by ELISA for supernatant concentration of TNF-α. Each data point is the mean of triplicate samples. Results show that TNF-α expression by PBMC was significantly increased relative to unstimulated control cultures as a result of PHA stimulation; and was suppressed by astaxanthin, but not GB treatment. As shown in Table 2, the combined treatment with both astaxanthin and GB suppressed expression of this pro-inflammatory cytokine below that of astaxanthin alone.

TABLE 2

| Stimulation | TNF-alpha | Standard |
|---|---|---|
| Conditions | pg/ml | Deviation |
| Unstimulated | 164.52 | 21.73 |
| PHA, 50 μg/ml | 1676.75 | 103.57 |
| Astx 10⁻⁶ M | 781.34 | 186.17 |
| BN52021 10⁻⁴ M | 1689.21 | 218.15 |
| Astx 10⁻⁶ M + | 225.76 | 52.97 |
| BN52021 10⁻⁴ M | | |

### EXAMPLE 3

### Compositions Containing A Biflavonoid Ginkgolide, Astaxanthin, Vitamin C, And/Or An NSAID Suppress Onset Of Alzheimer's Disease

Elevation of intracellular cAMP increases the recovery of APP alpha, the physiological alpha-secretase-derived product of beta APP processing, and concomittantly lowers the production of the pathogenic beta/gamma-secretase-derived A beta fragment (A42). The pathogenesis of Alzheimer's disease correlates with altered production, aggregation and deposition in neuronal tissue of of the A peptide, a proteolytic fragment of 40-42 residues derived from APP. The longer isoform, A42, is selectively increased in the disease and its presence promote production of beta-amyloid deposits. Beta amyloid in turn induces free radical production, increased glucose uptake, apoptosis and death of nerve cells. Extract of Ginkgo biloba (EGb 761) inhibits, in a dose-dependent manner, the formation of beta-amyloid-derived diffusible neurotoxic soluble ligands (ADDLs) involved in the pathogenesis of Alzheimer's disease. The mechanism for this protective effect involves elevation of neuronal cAMP which occurs as a result of the cAMP phosphodiesterase-inhibitory properties of the biflavonoid components of Ginkgo biloba.

NSAIDs ibuprofen, indomethacin and sulindac sulphide preferentially decrease the highly amyloidogenic A42 peptide (the 42-residue isoform of the amyloid-peptide) produced from a variety of cultured cells by as much as 80% independently of COX activity. Significant gastrointestinal and renal toxicity associated with long-term COX-1 inhibition limit the clinical utility of current NSAIDS as A42-lowering agents. Because the A42 effect is independent of COX activity, compounds (e.g., the combinations described herein) with optimized A42 reduction and little to no inhibition of COX-1 activity are useful for the prevention or alleviation of symptoms associated with Alzheimer's Disease. Such agents represent a new generation of ‘anti-amyloid’ drugs that selectively target production of the highly amyloidogenic A42 species without inhibiting either COX activity or the vital physiological functions.

Sustained high dosage, non-steroidal, anti-inflammatory drugs (NSAIDs) inhibit onset of Alzheimers disease, but the dosage required to suppress the disease is toxic.

Biflavonoid components of ginkgo biloba represent a new generation of anti-amyloid drugs which, when used in combination with NSAIDs lower the effective NSAID dosage to subtoxic levels, thereby enabling them to be used to prevent Alzheimer's disease at little or no risk to the general health of the patient.

Astaxanthin and vitamin C contribute to suppression of alzheimers disease in a manner synergistic with combiniations of ginkgo biflavoinoids by suppressing disease associated inflammation, primarily as free radical scavengers.

The compositions described herein are useful to prevent onset of Alzheimers disease by inhibiting formation of Beta amyloid plaques as a result of the combined action of the NSAIDs ibuprofen, indomethacin and sulindac sulphide, (and/or other drugs which act through COX-2 inhibition); plus the biflavonoid ginkgolides: amentoflavone, bilobetin, sequoiaflavone, ginkgetin and isoginkgetin. Inflammation associated with Alzheimers is suppressed by combining NSAID+ginkgolide formulations with astaxanthin and vitamin C. The combined action of the lipid-soluble carotenoid (principally astaxanthin) with vitamin C and one or more components of a Ginkgo biloba extract, in combination with NASAIDs mediates prevention or suppression of disease-associated inflammation. The combination drug therapy described herein utilizes astaxanthin and/or its derivatives; and a gingkolide composition, which contains cAMP phosphodiesterase-inhibitory capabilities.

### EXAMPLE 4

### Compositions Of Ginkgolides, Astaxanthin, Plus Vitamin C, Potentiate Anti-asthmatic Effects Of Cetirizine

Cetirizine compounds, e.g., Zyrtec™ (cetirizine hydrochloride), inhibit histamine release by mast cells. Histamine release occurs when mast cells are stimulated, e.g., when antibodies interact with their surface H1 receptors (H1R). Selective inhibition of H1R by Zrytec prevents downstream events which include intracellular calcium ion release and calcium uptake and protein kinase C translocation. H1 inhibition inhibits these effects and also promotes the activation of adenylate cyclase and the resulting accumulation of cAMP.

Components of Ginkgo biloba include terpene antagonists of PAF receptors (PAFR) which synergize with cetirizine and other histamine release blockers in reducing the calcium signal (a consequence of PAFR stimulation). Biflavonoid ginkgolides further reduce the effective dosage of cetirizines by their inhibition of cAMP phosphodiesterase, an effect which allows augmented accumulation of cAMP.

Astaxanthin also potentiates the effect of cetirizines. Histamine release from mast cells is significantly reduced by antioxidants, and astaxanthin further contributes to potentiation of the pharmacological activity of cetirizines.

The compositions described herein are useful to augment the therapeutic activity of cetirizines such as Zyrtec™, while reducing its effective dosage. For example, such composition contain a cetirizine compound plus terpene trilactones, such as Gingkolide A, Gingkolide B, Gingkolide C, Gingkolide J, Gingkolide M and bilobalide; or the biflavonoid ginkgolides: amentoflavone, bilobetin, sequoiaflavone, ginkgetin and isoginkgetin. The combined action of the lipid-soluble carotenoid (e.g., astaxanthin) with vitamin C and one or more components of a Ginkgo biloba extract, mediates prevention or suppression of disease-associated inflammation. The combination drug therapy described herein utilizes astaxanthin and/or its derivatives; and a gingkolide composition, which contains cAMP phosphodiesterase-inhibitory as well as antioxidant capabilities.

Cetirizine compounds such as Zyrtec™ are antihistamines useful in general treatment of allergies, especially seasonal or perennial rhinitis and chronic urticaria. The risk of toxicity associated with such compounds is substantially increased in individuals with kidney impairment, in particular geriatric patients. The combination drug therapy regimen (e.g., cetirizine administered with astaxanthin and/or a gingkolide), reduces the effective dosage necessary for a beneficial clinical outcome.

The lipid antioxidant astaxanthin and the terpene and biflavonoid components of ginkgo biloba synergize with a cetirizine such as Zyrtec™ to reduce H1-mediated histamine release by mast cells and other tissue. The synergistic effect of this combination permits a reduction in the effective dosage of a cetirizine needed to achieve a desired therapeutic outcome, thereby reducing adverse side effects of a cetirizine compound.

When cells were cultured in the presence of Ginkgolide B (GB) plus cetirizine, the PMA/Ionomycin-induced expression of the T cell activation antigen CD25 was suppressed to levels below that mediated by either GB or Zrytec alone.

Astaxanthin or astaxanthin plus a cetirizine together was administered to an allergy patient. Astaxanthin alone did not result in alleviation of allergy symptoms. However, the therapeutic effect of the combination (an antioxidant such as astaxanthin and cetirizine) exceeded that of either agent alone (as measured by reduction of allergry symptoms such as itching).

### EXAMPLE 5

### Suppression Of Lymphocyte Activation By Citirazene And Azalestine

Experiements were carried out to determine whether the immunoregulatory capacity of two commonly-used H1-inhibitory antihistamines: cetirizine dihydrochloride (CTZ/Zyrtec) and azelastine (AZE/Astelin) is potentiated by the platelet activating factor receptor (PAFR) antagonist and free radical scavenger Ginkgolide B (GB). For these studies, peripheral blood mononuclear cells (PBMC) from asthma patients, which were cultured 24 hours with either 50 μg/ml PHA or PHA plus selected dosages of each drug were analyzed by 3-color flow cytometry for expression of CD25+ and HLA-DR+ on CD3+ (T cells). The results shown in Table 3 are reported as stimulation indices (SI) of %CD3+CD25+ cells in cultures treated with PHA alone to %CD3+CD25+ cells in each drug-supplemented culture. Each drug was first evaluated independently over a 3-log dose range from 10⁻⁸-10⁻⁶ M. Maximal suppression of activation was observed at 10⁻⁸ M, where CTZ caused a 29% decrease in SI for CD25+ (p=0.024); and 53% for HLA-DR (p=0.009); with AZE resulting in decreases of 19% for CD25+ (p=0.33); and 45% for HLA-DR (p=0.001); and GB 10⁻⁸ M suppressing HLA-DR+ by 39% (p=0.01). When compared to effects at 10⁻⁸ M, each drug at 10⁻⁷ M showed reduced capacity to independently suppress PHA-mediated induction of the two activation antigens. However at this concentration, GB was observed to augment the capacity of CTZ to suppress expression of CD25+ (p=0.003) and HLA-DR (p=0.004). The suppressive effect of AZE at 10⁻⁷ was also potentiated by GB at the same concentration in the case of CD25+ (p=0.014) and HLA-DR (p=0.000). The data indicated that GB improved the pharmacological activity of CTZ and AZE at a concentration of 10⁻⁷ M for each of the three components. These data indicate that GB-augmented antihistamine formulations are useful to alleviate a symptom of asthma-associated inflammation, e.g., abnormal T cell activation.

TABLE 3
Effect of cetirizine/Zyrtec (CTZ), or azalestene (AZE) on induction of CD25+
(CD3+CD25+) and HLA-DR+ (CD3+HLA-DR+) T lymphocytes in human peripheral
blood mononuclear cells (PBMC)

| | | P vs N | | | P vs N | |
|---|---|---|---|---|---|---|
| Culture | SI CD25 | Stim | subjects | SI HLA-DR | Stim | subjects |
| Unstimulated | 0.09 ± 0.03 | 0.000 | 20 | 0.450 ± 0.09 | 0.000 | 6 |
| Stimulated | 1.00 ± 0.00 | — | 20 | 1.00 ± 0.00 | — | 5 |
| CTZ 10⁻⁸ M | 0.71 ± 0.12 | 0.024 | 8 | 0.47 ± 0.13 | 0.009 | 5 |
| CTZ 10⁻⁷ M | 0.88 ± 0.14 | 0.060 | 20 | 0.58 ± 0.11 | 0.010 | 5 |
| CTZ 10⁻⁶ M | 0.93 ± 0.06 | 0.141 | 20 | 0.70 ± 0.04 | 0.001 | 5 |
| AZE 10⁻⁸ M | 0.81 ± 0.08 | 0.033 | 8 | 0.55 ± 0.09 | 0.001 | 5 |
| AZE 10⁻⁷ M | 0.88 ± 0.08 | 0.092 | 19 | 0.56 ± 0.10 | 0.006 | 5 |
| AZE 10⁻⁶ M | 1.13 ± 0.15 | 0.210 | 17 | 0.61 ± 0.13 | 0.018 | 5 |
| GB 10⁻⁸ M | 0.81 ± 0.14 | 0.105 | 8 | 0.61 ± 0.10 | 0.010 | 5 |
| GB 10⁻⁷ M | 1.18 ± 0.21 | 0.196 | 14 | 0.62 ± 0.18 | 0.051 | 5 |
| GB 10⁻⁶ M | 0.94 ± 0.12 | 0.308 | 15 | 0.72 ± 0.18 | 0.100 | 5 |
| GB 10⁻⁸ M + CTZ 10⁻⁸ M | 0.79 ± 0.08 | 0.038 | 8 | 0.62 ± 0.13 | 0.020 | 5 |
| GB 10⁻⁸ M + CTZ 10⁻⁷ M | 0.88 ± 0.08 | 0.096 | 8 | 0.75 ± 0.13 | 0.630 | 5 |
| GB 10⁻⁸ M + AZE 10⁻⁸ M | 0.86 ± 0.09 | 0.091 | 8 | 0.61 ± 0.04 | 0.001 | 5 |
| GB 10⁻⁸ M + AZE 10⁻⁷ M | 0.71 ± 0.12 | 0.024 | 7 | 0.53 ± 0.17 | 0.035 | 4 |
| GB 10⁻⁷ M + CTZ 10⁻⁸ M | 0.73 ± 0.07 | 0.002 | 8 | 0.64 ± 0.10 | 0.012 | 5 |
| GB 10⁻⁷ M + CTZ 10⁻⁷ M | 0.65 ± 0.09 | 0.003 | 8 | 0.51 ± 0.11 | 0.004 | 5 |
| GB 10⁻⁷ M + AZE 10⁻⁸ M | 0.73 ± 0.12 | 0.033 | 8 | 0.62 ± 0.11 | 0.014 | 5 |
| GB 10⁻⁷ M + AZE 10⁻⁷ M | 0.71 ± 0.10 | 0.014 | 8 | 0.50 ± 0.04 | 0.000 | 5 |

### Example 6

### Effects Of Astaxanthin and Ginkgolide B On T Lymphocyte Activation

Experiments were carried out to determine whether formulations based on the platelet activating factor receptor (PAFR) antagonist and free radical scavenger Ginkgolide B (GB) in combination with the antioxidant carotenoid astaxanthin (ASX) suppress T cell activation in the same dose range as two commonly-used antihistamines: cetirizine dihydrochloride (CTZ/Zyrtec) and azelastine (AZE/Astelin). Peripheral blood mononuclear cells (PBMC) from asthma patients were cultured 24 hours with either 50 μg/ml PHA or PHA plus selected dosages of each drug and analyzed by 3-color flow cytometry for expression of CD25+ on CD3+ (T cells). Results are reported as stimulation indices (SI) of %CD3+CD25+ cells in cultures treated with PHA alone to %CD3+CD25+ cells in each drug-supplemented culture. Formulations which significantly reduced SI of PHA-treated cells ranked in order of increasing magnitude of suppression are as follows: ASX 10⁻⁷ M<GB 10⁻⁸M+ASX 10⁻⁸M<GB 10⁻⁸M<GB10⁻⁷M+ASX 10⁻⁷M<GB 10⁻⁸M+ASX 10⁻⁷M ASX<CTZ 10⁻⁵M<GB 10⁻⁶M<GB 10⁻⁷M+ASX 10⁻⁸M<AZE 10⁻⁵M. The data indicate that suppression of T cell activation below fully-stimulated values by GB, ASX and their combinations was comparable and for some combinations better than that mediated by CTZ and AZE.

The studies were carried out as follows.

Patients

Subjects for this study included 12 patients diagnosed with atopic asthma, 7 male and 5 female, ranging in age from 21 to 40 years (mean 28±1.8 years). Disease duration ranged from 2 to 12 years. Atopy was defined on the basis of one or more positive skin prick tests to a range of 20 allergens. None of the patients had received systemic therapy for at least 6 weeks prior to blood collection. The mean serum IgE was 335 (170-480) IU/ml.

Cell Cultures

Venous blood for each subject was collected in polyethylene tubes containing EDTA during a one hour morning time interval. PBMC were separated by Ficoll-paque (Pharmacia, Uppsala, Sweden) density gradient centrifugation. Cells were washed and suspended in RPMI 1640 medium (Gibco BRL, Gaithersburg, Md.) at density of 1×10⁶ cells/ml. PBMC were stimulated with 50 μg/ml Phytohemaglutinin (PHA) (Sigma Immunopharmaceuticals, St. Lous Mo.), or PHA plus 10⁻⁸-10⁻⁵ M astaxanthin (Natural Alternatives International (NAI) Inc., San Marcos Calif.); or ginkgolide B 10⁻⁸-10⁻⁶ M (NAI San Marcos Calif.); or selected combinations of ASX plus GB. Comparison of ASX and GB effects on T cell activation were made with two other pharmacological agents with anti-asthmatic properties by treating cells with 10⁻⁷-10⁻⁴ M cetirizine dihydrochloride (Pfizer Pharmaceuticals, Norwich Conn.); or 10⁻⁷-10⁻⁴ M azalestine hydrochloride (Wallace Pharmaceuticals, Somerset N.J.), followed by evaluation of cultures for the same biological endpoints as ASX/GB-treated cells. Each reagent was added at the outset of a 24 hours culture period, followed by harvest of cellular fraction for immunophenotyping studies.

Flow Cytometric Analysis

Cells harvested from cultures by centrifugation were incubated for 30 min at 4° C. with 10 μl each of flourescein-isothiocyanate (FITC)-CD3 and phycoerythrin (RD1)-CD25 conjugated monoclonal antibodies (mAb) (Dakopatts, A/S, Glostrup, Denmark), followed by fixation with paraformaldehyde. Two-color Flow cytometry was conducted using a Coulter Epics XL automated flow cytometer (Coulter Scientific, Hialeah, Fla., USA). Isotypic controls for the monoclonal antibodies (mAb) used to detect antigens of interest were established for each cell preparation. Positive analysis regions for cells expressing specific surface markers were set against controls and specific binding of fluorophore-conjugated mAb was analyzed by cytofluorography according to standard methods recommended by the manufacturer. Lymphocyte subpopulations were identified by position on forward and side scatter plots and live-gated. Expression of each antigen was reported as percentage cells positive for a particular T cell subpopulation defined by expression of CD3 (T lymphocyte marker) plus CD25, plus or minus standard error.

Statistical Analysis

Statistical analysis was performed using an independent t-test. All statistical analyses were performed using the SPSS for Windows statistical package (Norusis/SPSS, Inc.). A value of p<0.05 was considered statistically significant

T Lymphocyte Activation

Culture of PBMC for 24 hours with 50 μg/ml of the immunostimulatory lectin PHA resulted in significant activation of T lymphocytes, measured as increased percentage of CD3+CD25+ cells versus unstimulated cultures (Table 3). The capacity of formulations evaluated in this study to suppress immune activation was measured as a stimulation index (SI), defined as the ratio of CD3+CD25+ cells in each test condition to CD3+CD25+ in cultures treated with PHA alone. Assigning fully-stimulated cultures an SI value of 1.00, we observed that 9 of the 26 candidate formulations resulted in significant (p<0.05) reduction in SI (Table 1).

Effects of Astaxanthin and GB on T Lymphocyte Activation

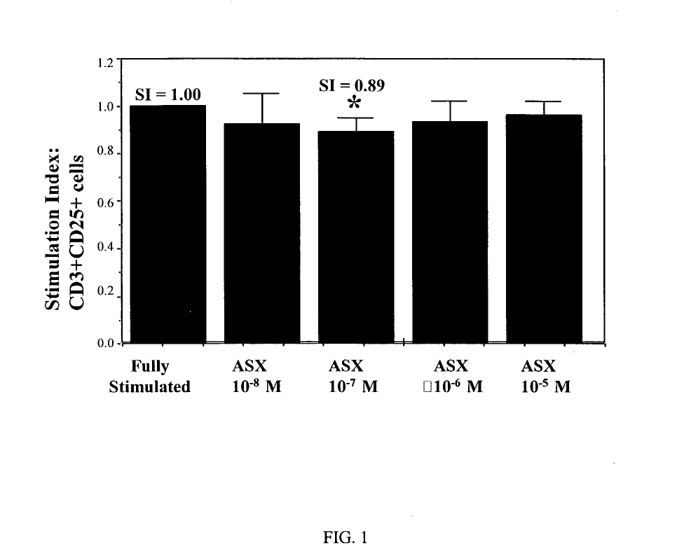

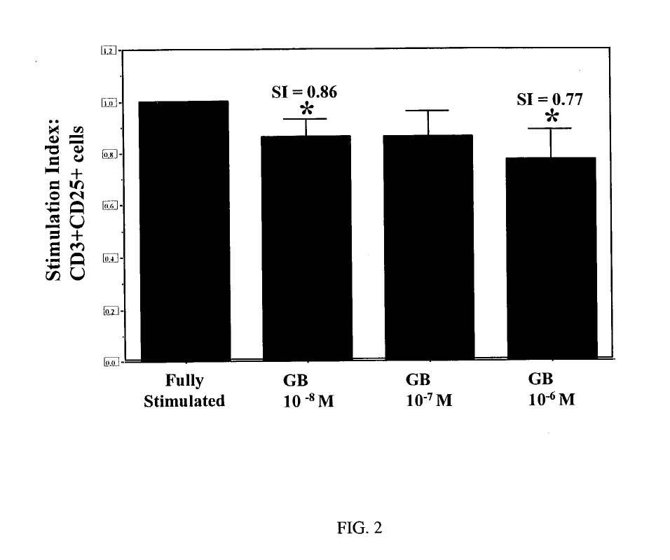

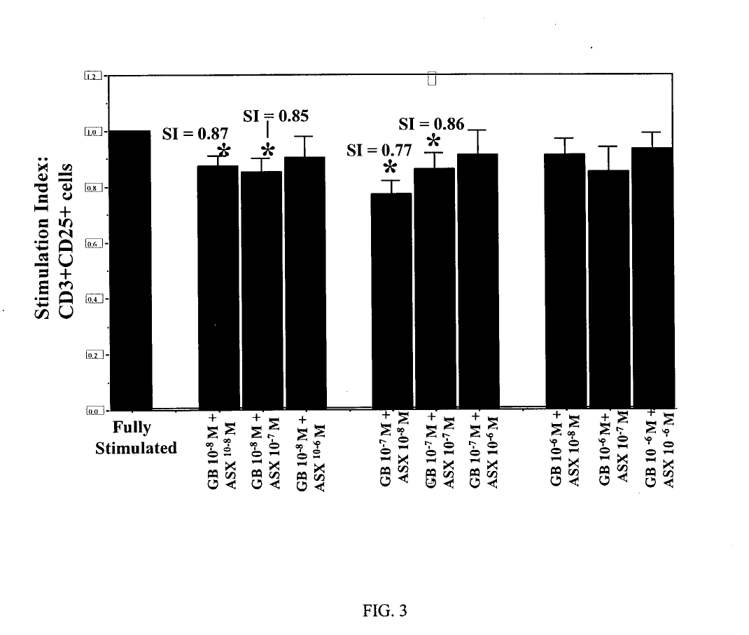

As shown in FIG. 1, stimulation indices for PHA-treated cells were suppressed significantly by astaxanthin at a concentration of 10⁻⁷ M (SI=0.89±0.06, p<0.034). Ginkgolide B significantly reduced SI of PHA-stimulated cells at dosages of 10⁻⁶M (SI=0.77±0.12, p=0.048); and 10⁻⁸M (SI=0.86±0.07, p=0.05) (FIG. 2). Combinations of these agents also significantly suppressed immune activation. These formulations included 10⁻⁷M GB in combination with 10⁻⁷M ASX (SI=0.86±0.06, p=0.037); 10⁻⁷M GB+10⁻⁸M ASX (SI=0.77±0.05, p=0.006); 10⁻⁸ M GB+10⁻⁷ M ASX (SI=0.85±0.05, p=0.015); and 10⁻⁸M GB+10⁻⁸M ASX (SI=0.87±0.06, p=0.040) (FIG. 3); and cells stimulated with a combination of 10⁻⁸M ASX plus 10⁻⁷M ginkgolide B, which suppressed induction of CD3+CD25+ cells to an SI of 0.77±0.05, significantly below the suppression mediated by 10⁻⁸M ASX alone acting on PHA-stimulated cultures (p=0.051) (FIGS. 1 and 3). Nevertheless treatment of cells with 10⁻⁷M GB+10⁻⁸M ASX failed to significantly suppress activation below 10⁻⁷M GB alone acting on PHA-treated cells (p=0.373) (FIGS. 2 and 3).

Effects of Cetirizine and Azelastine on T Lymphocyte Activation

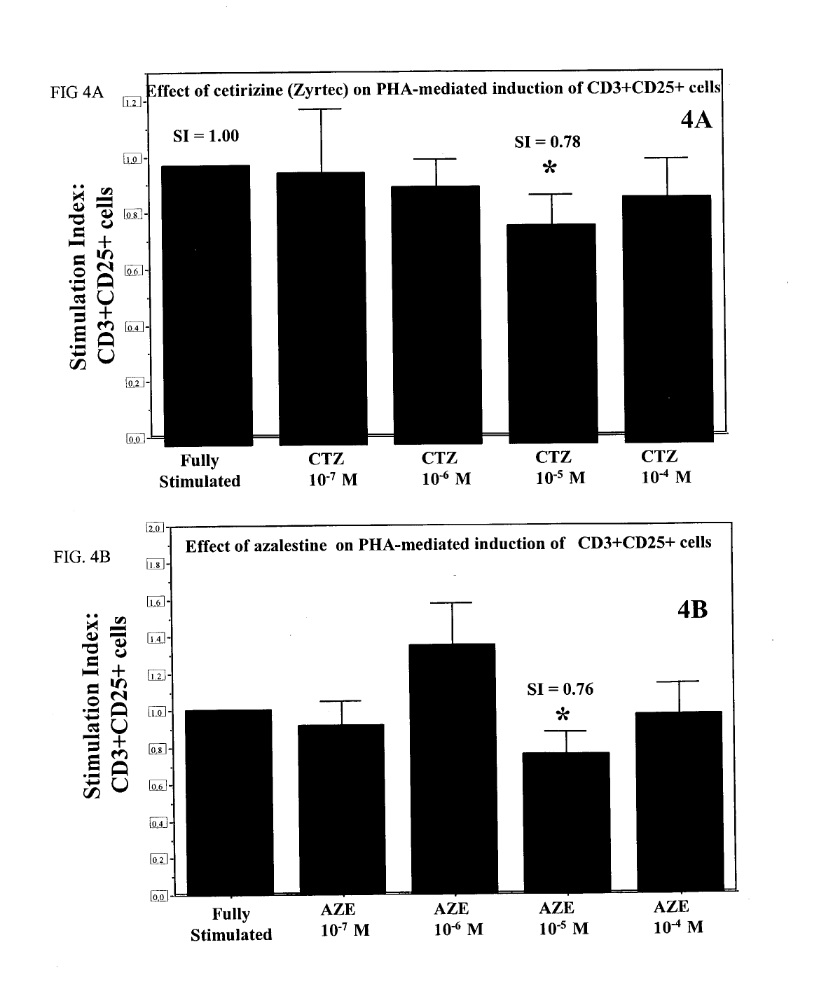

Two commonly-used anti asthmatic compounds, cetirizine dihydrochloride (Zyrtec, CTZ) and azelastine HCl (Astelin, AZE) were evaluated under the same conditions as ASX and GB for their ability to suppress T cell activation. Cells treated with PHA exhibited significant reduction in induction of CD3+CD25+ cells at a concentration of 10⁻⁵M for both CTZ(SI=0.78±0.11, p=0.05) (FIG. 4A); and AZE (SI=0.76±0.12, p=0.034) (FIG. 4B).

Combination Drug Therapy for Inhibition of T Cell Activation in Asthma Subjects

Asthma is associated with elevated expression in bronchoalveolar tissue of Th2 cytokines (IL-3, IL-5, and GM-CSF), which in turn upregulate eosinophil recruitment, activation, proliferation and differentiation, promoting tissue injury and fibrosis via an increased production of a variety of toxic metabolites. Histamine release blockers such as azalestine and cetirizine which treat the disease downstream from the underlying pathogenic T lymphocyte activity have been successful in partially alleviating its symptoms, but are often not as effective as agents which directly suppress abnormal T cell activation. Nevertheless since cellular signalling pathways which promote tissue damage in asthma, exert positive feedback and increase T cell activation, drugs which inhibit release or activity of inflammatory metabolites are also expected to exhibit immunosuppressive properties. Indeed, the HI receptor antagonist terfenadine is observed to inhibit proliferation and expression of IL-4 and IL-5 production by anti-CD3/-CD28 and PMA-activated human T cells in vitro. Since both of these Th2 cytokines are implicated as major factors in asthma pathogenesis, therapeutic effects of this drug are likely mediated at least in part by suppression of T cell activity.

Ginkgolide B and astaxanthin with azalestine and cetirizine were tested for the ability to suppress T cell activation in PHA-stimulated cultures of human PBMC taken from asthma patients. These experiments were designed with the recognition that suppression of T lymphocyte activation is not the primary mechanism by which each compound mediates its therapeutic effects. However, since T cell activity is a critical component of the cascade of signaling events resulting in the symptoms of asthma, T cell suppression represents a useful index to gauge the relative effectiveness of the pharmacological agents tested. Table 3 shows the effect of each stimulation condition on cells with respect to their ability to inhibit PHA-induced upregulation of the IL-2 receptor (CD25) on CD3+ cells (an index of T cell activation). When astaxanthin alone was added to PHA-treated cultures, significant suppression of T lymphocyte activation occurred at a concentration of 10⁻⁷M (FIG. 1); whereas SI values significantly lower than 1.00 (fully stimulated) were observed over a 3 log dose range of ginkgolide B, with SI values significantly less than 1.00 observed at GB concentrations of 10⁻⁸ M and 10⁻⁶ (FIG. 2). When combinations of ASX and GB were evaluated for their capacity to suppress T cell activation, four combinations of the compounds were observed to result in significant reduction in PHA-mediated induction of CD3+CD25+ cells; with an optimal combination occurring at a concentration of 10⁻⁸ M ASX plus 10⁻⁷ M GB (FIG. 3). Mechanisms contributing to suppression of T cell activation by ASX, GB and their combination are likely a consequence of the major biochemical properties of each compound acting together. Reactive oxygen species (ROS) are substantially upregulated by T lymphocytes during PHA-mediated activation; moreover blocking this enhancement with antioxidants alters the activation process.

Although previous studies of cetirizine suggest that it has no significant effect on T cells, the data described herein indicate that at an optimal concentration of 10⁻⁵ M, it will suppress at least those aspects of T cell activation involving expression of CD25 (FIG. 4A). Cetirizine also displays an ability to downregulate aspects of T cell activation related to chemotaxis. The present results indicate that astaxanthin and ginkgolide B act in concert to mediate antiasthmatic effects as well or better than currently-used medications. Compositions containing the combination of compounds described herein reduce inflammation (e.g., by inhibiting T cell activation) with little or none of the side effects associated with conventional anti-inflammatory medicaments. When these compositions are administered in conjunction with conventional anti-inflammatory drugs, less of the conventional drug is required to achieve the same or similar therapeutic benefit, thereby reducing undesirable side effects associated with the conventional drug.

Other embodiments are within the following claims.

What is claimed is:

1. A composition comprising a lipid soluble antioxidant and a water soluble antioxidant, wherein said lipid soluble antioxidant is a carotenoid compound.

2. The composition of claim 1, wherein said carotenoid compound is astaxanthin or an ester thereof.

3. The composition of claim 1, wherein said water soluble antioxidant is ascorbic acid e antioxidant is ascorbic acid.

4. The composition of claim 1, wherein said water soluble antioxidant is a ginkgolide.

5. The composition of claim 4, wherein said ginkgolide comprises a terpene trilactone selected from the group consisting of Gingkolide A, Gingkolide B, Gingkolide C, Gingkolide J, Gingkolide M, and bilobalide.

6. The composition of claim 4, wherein said ginkgolide comprises Egb 761.

7. The composition of claim 4, wherein said ginkgolide comprises BN50730.

8. The composition of claim 4, wherein said ginkgolide comprises BN52021.

9. The composition of claim 1, wherein said composition further comprises an histamine release inhibitor.

10. The composition of claim 9, wherein said histamine release inhibitor is selected from the group consisting of a cetirizine compound and an azelastine compound.

11. A composition comprising an asaxanthin compound, a ginkgolide compound, and an ascorbic acid compound.

12. A method of inhibiting activation of an immune cell, comprising contacting said immune cell with the composition of claim 1.

13. The method of claim 12, wherein said immune cell is a T cell.

14. The method of claim 12, wherein said immune cell is a mast cell.

15. A method of alleviating a symptom of an inflammatory disease, comprising administering to a mammal suffering from or at risk of developing said disease the composition of claim 1.

16. The method of claim 15, wherein said composition is administered systemically.

17. The method of claim 15, wherin said composition is administered locally.

18. The method of claim 17, wherein said composition is administered by directly contacting an inflammed tissue with the composition of claim 1.

19. The method of claim 18, wherein said tissue is dermal tissue.

20. The method of claim 18, wherein said tissue is pulmonary tissue.

21. The method of claim 18, wherein said tissue bronchoalveolar tissue.
